(19) United States
(12) Patent Application Publication
(10) Pub. No.: US 2011/0098759 A1
(43) Pub. Date: Apr. 28, 2011

(54) EXPANDABLE DEVICE FOR BONE MANIPULATION

(75) Inventor: Hai H. Trieu, Cordova, TN (US)

(73) Assignee: KYPHON SARL, Neuchatel (CH)

(21) Appl. No.: 12/607,829

(22) Filed: Oct. 28, 2009

(51) Int. Cl. A61B 17/88; A61B 17/58

(52) U.S. Cl. 606/86 R; 606/90

(57) ABSTRACT

An expandable bone tamp for performing a minimally invasive surgical procedure includes a coil connected between an inner shaft and an outer shaft. Rotating the inner shaft relative to the outer shaft can then cause the coil to expand or collapse around the inner shaft. The coil allows large expansion forces to be generated by a structure Thant can pass through a small cannula. The rotation of the inner shaft relative to the outer shaft can be performed manually (e.g., via a crank handle) for good tactile control, or can be performed using a motor or other assist mechanism to increase the expansion force output. The coil can either be withdrawn after use, or can be left in the patient to provide additional post-procedure support.

### FIELD OF THE INVENTION

The invention relates to a system and method for performing a surgical procedure, and in particular, to a bone manipulation device that can be deployed percutaneously and generate large expansion forces.

### BACKGROUND OF THE INVENTION

A minimally invasive procedure is a medical procedure that is performed through the skin or an anatomical opening. In contrast to an open procedure for the same purpose, a minimally invasive procedure will generally be less traumatic to the patient and result in a reduced recovery period.

However, there are numerous challenges that minimally invasive procedures present. For example, minimally invasive procedures are typically more time-consuming than their open procedure analogues due to the challenges of working within a constrained operative pathway. In addition, without direct visual feedback into the operative location, accurately selecting, sizing, placing, and/or applying minimally invasive surgical instruments and/or treatment materials/devices can be difficult.

For example, for many individuals in our aging world population, undiagnosed and/or untreatable bone strength losses have weakened these individuals' bones to a point that even normal daily activities pose a significant threat of fracture. In one common scenario, when the bones of the spine are sufficiently weakened, the compressive forces in the spine can cause fracture and/or deformation of the vertebral bodies. For sufficiently weakened bone, even normal daily activities like walking down steps or carrying groceries can cause a collapse of one or more spinal bones. A fracture of the vertebral body in this manner is typically referred to as a vertebral compression fracture. Other commonly occurring fractures resulting from weakened bones can include hip, wrist, knee and ankle fractures, to name a few.

Fractures such as vertebral compression fractures often result in episodes of pain that are chronic and intense. Aside from the pain caused by the fracture itself, the involvement of the spinal column can result in pinched and/or damaged nerves, causing paralysis, loss of function, and intense pain which radiates throughout the patient's body. Even where nerves are not affected, however, the intense pain associated with all types of fractures is debilitating, resulting in a great deal of stress, impaired mobility and other long-term consequences. For example, progressive spinal fractures can, over time, cause serious deformation of the spine (“kyphosis”), giving an individual a hunched-back appearance, and can also result in significantly reduced lung capacity and increased mortality.

Because patients with these problems are typically older, and often suffer from various other significant health complications, many of these individuals are unable to tolerate invasive surgery. Therefore, in an effort to more effectively and directly treat vertebral compression fractures, minimally invasive techniques such as vertebroplasty and, subsequently, kyphoplasty, have been developed. Vertebroplasty involves the injection of a flowable reinforcing material, usually polymethylmethacrylate (PMMA—commonly known as bone cement), into a fractured, weakened, or diseased vertebral body. Shortly after injection, the liquid filling material hardens or polymerizes, desirably supporting the vertebral body internally, alleviating pain and preventing further collapse of the injected vertebral body.

Because the liquid bone cement naturally follows the path of least resistance within bone, and because the small-diameter needles used to deliver bone cement in vertebroplasty procedure require either high delivery pressures and/or less viscous bone cements, ensuring that the bone cement remains within the already compromised vertebral body is a significant concern in vertebroplasty procedures. Kyphoplasty addresses this issue by first creating a cavity within the vertebral body (e.g., with an inflatable balloon) and then filling that cavity with bone filler material. The cavity provides a natural containment region that minimizes the risk of bone filler material escape from the vertebral body. An additional benefit of kyphoplasty is that the creation of the cavity can also restore the original height of the vertebral body, further enhancing the benefit of the procedure.

However, in many instances, a fractured vertebra “sets” in its fractured condition, as the bone partially heals in its compressed state. In such instances, restoration of the vertebral body height can require more lifting force than can be provided by conventional kyphoplasty bone tamps. Consequently, the kyphosis caused by the vertebral compression fracture is not corrected, and the problems associated with such kyphosis (e.g., hunched posture, reduced lung capacity, increased likelihood of adjacent vertebral fracture) remain.

Accordingly, it is desirable to provide surgical tools and techniques that provide more effective vertebral body height restoration during the treatment of compression fractures.

### SUMMARY OF THE INVENTION

By incorporating an expandable coil structure into a percutaneously deployable bone tamp, the bone tamp can apply significant lifting forces to the endplates of a collapsed vertebra, thereby enhancing the likelihood of height restoration of the vertebral body during a kyphoplasty procedure.

In one embodiment, an expandable bone tamp can include a coil connected between an outer shaft and an inner shaft that is rotatably disposed in the outer shaft. Therefore, by rotating the inner shaft relative to the outer shaft, the coil can be expanded (i.e., unwound from around the inner shaft) or collapsed (i.e., wound more tightly around the inner shaft) as desired. Various types of holding mechanisms can be used to selectively or automatically maintain the size of the coil when the inner shaft is not being actively rotated relative to the outer shaft.

In various embodiments, the rotation of the inner shaft relative to the outer shaft can be performed directly on the inner shaft (e.g., a crank handle attached to the inner shaft). In various other embodiments, the rotation can be performed by mechanical, electrical, and/or hydraulic systems that can provide a greater input torque (e.g., a motor or gear train).

In some embodiments, the coil can be formed of a shape memory or spring material (e.g., Nitinol or spring steel). In other embodiments, the coil can be formed of a non-resilient material (e.g., aluminum oxide or gold) that deforms plastically in response to the rotational loading of the inner and outer shafts.

In various embodiments, the coil turns can have a material cross section in the shape of a rectangle, circle, triangle, trapezoid, or any other shape providing a desired bone interaction. In various other embodiments, the coil turns can be configured to exhibit different expansion properties, such that coil expansion results in a non-cylindrical profile (e.g., outwardly tapering, inwardly tapering, ovoid, peanut-shaped, etc.).

In various other embodiments, a surgical kit can include an expandable bone tamp that includes a coil connected between an outer shaft and an inner shaft that is rotatably disposed in the outer shaft. In some embodiments, the kit can further include a cannula sized to allow passage of the coil when the coil is tightly wrapped around the inner shaft. In other embodiments, the kit can further include instructions for performing a surgical procedure using the expandable bone tamp.

In various other embodiments, a surgical procedure can be performed by placing in a target bone (e.g., a collapsed vertebra) an expandable bone tamp that includes a coil connected between an outer shaft and an inner shaft that is rotatably disposed in the outer shaft. The inner shaft can then be rotated in a first direction relative to the outer shaft to expand the coil, thereby creating a cavity in the bone and optionally restoring an original cortical wall profile for the bone (e.g., restoring the height of the collapsed vertebra). The cavity can then be filled with bone filler material to provide support for the bone.

In some embodiments, the coil can be removed from the bone after cavity creation by rotating the inner shaft in a second direction relative to the outer shaft to wind the coil tightly around the inner shaft, and then withdrawing the coil through the cannula. In other embodiments, the coil can be detached from the expandable bone tamp and left in the cavity as the bone filler material is delivered.

In various other embodiments, the surgical procedure can further include placing a second expandable bone tamp in the target bone (e.g., a kyphoplasty procedure using bilateral access). Then one of the expandable bone tamps can be used to provide support for the bone while the other is withdrawn and bone filler material delivery is taking place.

As will be realized by those of skilled in the art, many different embodiments of an expandable bone tamp incorporating a coil, along with systems, kits, and/or methods of using such an expandable bone tamp according to the present invention are possible. Additional uses, advantages, and features of the invention are set forth in the illustrative embodiments discussed in the detailed description herein and will become more apparent to those skilled in the art upon examination of the following.

### BRIEF DESCRIPTION OF THE DRAWINGS

FIGS. 1A-1E show an exemplary expandable bone tamp that incorporates a coil structure that can be actively expanded and collapsed.

FIGS. 2A-2B show sample coil material cross sections.

FIGS. 3A-3B show sample alternative coil expansion profiles.

FIG. 4 shows a kit that includes an expandable bone tamp that incorporates a coil structure that can be actively expanded and collapsed.

FIGS. 5A-5H show an exemplary kyphoplasty procedure using an expandable bone tamp incorporating a coil structure for bone manipulation.

FIGS. 6A-6C show an exemplary bilateral kyphoplasty procedure using expandable bone tamps incorporating coil structures for bone manipulation.

### DETAILED DESCRIPTION

By incorporating an expandable coil structure into a percutaneously deployable bone tamp, the bone tamp can apply significant lifting forces to the endplates of a collapsed vertebra, thereby enhancing the likelihood of height restoration of the vertebral body during a kyphoplasty procedure.

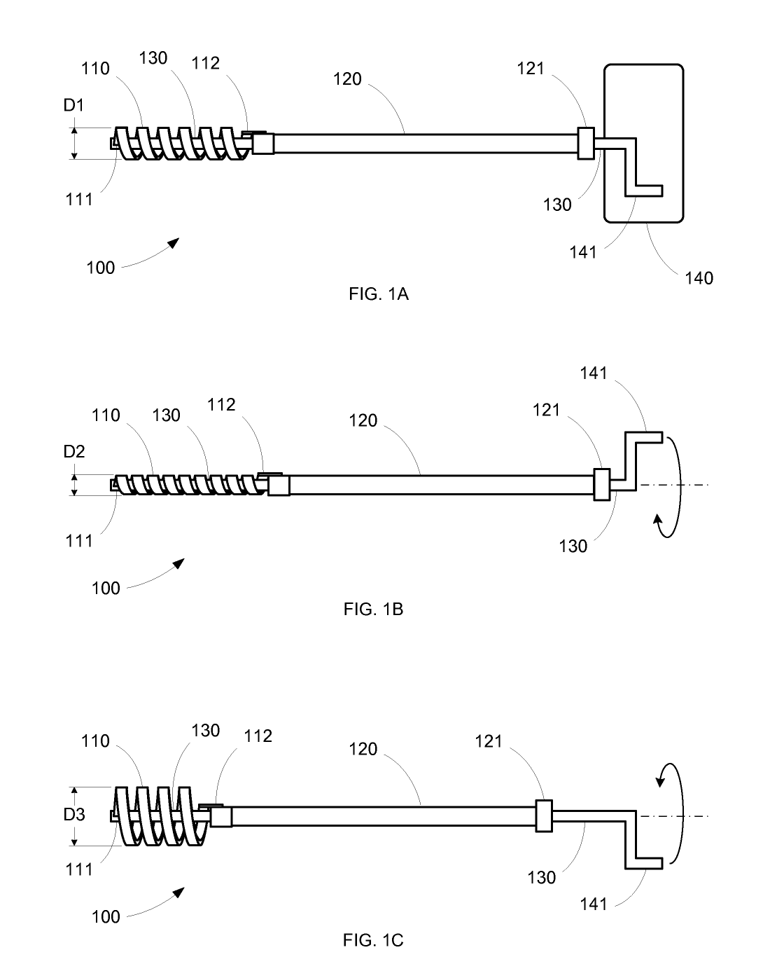

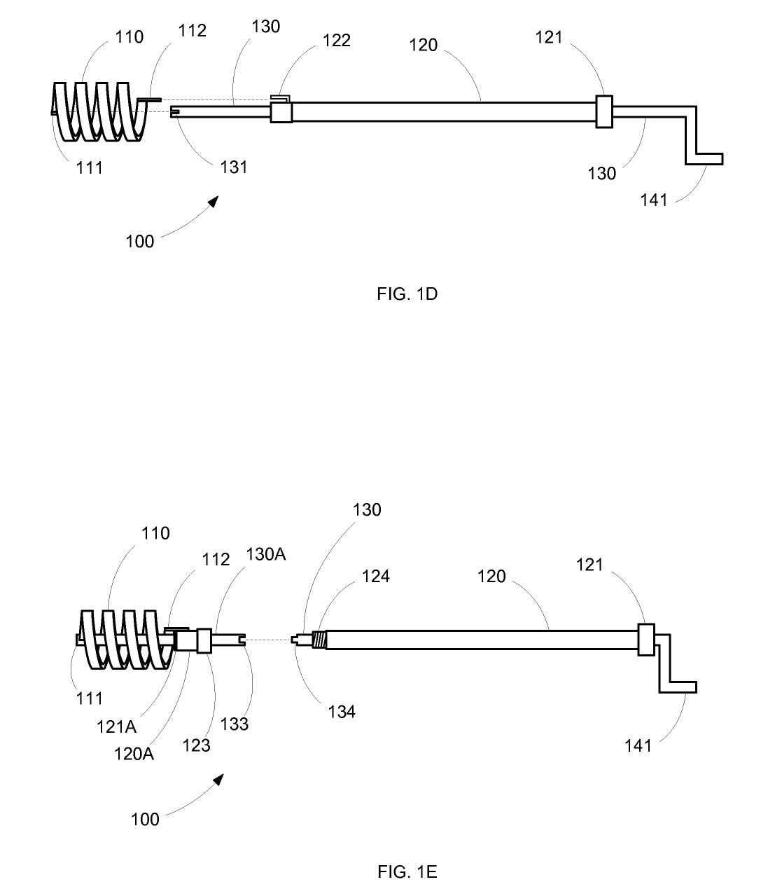

FIG. 1A shows an embodiment of an expandable bone tamp 100 that can be used in a kyphoplasty procedure. Expandable bone tamp 100 includes a coil 110, an outer shaft 120, and an inner shaft 130 rotatably disposed within outer shaft 120 (i.e., inner shaft 130 can rotate within outer shaft 120). One end 111 of coil 110 is coupled to inner shaft 130, and the other end 112 of coil 110 is coupled to outer shaft 120. Expandable bone tamp 100 further includes a holding mechanism 121 and a rotational actuator 140.

Holding mechanism 121 is a mechanism for controllably maintaining a relative rotational orientation between inner shaft 130 and outer shaft 120 (i.e., allowing the user to rotate inner shaft 130 with respect to outer shaft 120, but preventing relative rotation otherwise). In various embodiments, holding mechanism 121 can be a manually actuated system (e.g., a releasable clamp or locking mechanism). In various other embodiments, holding mechanism 121 can be an automatic system (e.g., a clamping shaft collar or a ratchet). Note that while depicted at the proximal end of outer shaft 120 for exemplary purposes, in various other embodiments, holding mechanism 121 can be at any location, including any location along (or within) outer shaft 120, within rotational actuator 140, or along inner shaft 130.

Finally, rotation mechanism 140 is an actuation mechanism for rotating inner shaft 130 with respect to outer shaft 120. For exemplary purposes, rotational actuator 140 is depicted and described below as a crank handle 141 for directly rotating inner shaft 130 within outer shaft 120. However, in various other embodiments, rotational actuator 140 can be any system for rotating inner shaft 130 with respect to outer shaft 120 (e.g., an electric or battery-powered motor, a linear-to-rotary motion converter (such as a ball screw or lead screw), a belt or cable and pulley system, and a gear train, among others). In some embodiments, rotational actuator 140 can be operated remotely from inner shaft 130 and outer shaft 120 (e.g., remote control of a motor via wires, remote torque application via a linkage or gear train, or remote torque application via a rotation cable (e.g., push-pull cable), among others).

Note that the particular performance parameters of expandable bone tamp 100 can determine the appropriate implementation of rotational actuator 140. For example, if tactile feedback and direct control over the actuation of expandable tamp 100 is deemed critical, then a rotary mechanism 140 consisting of crank handle 141 connected directly to inner shaft 130 can be used. Alternatively, if increased actuation force is required, then a powered motor and/or gear train may be a more appropriate choice.

As noted above, coil 110 is coupled between inner shaft 130 and outer shaft 120. Therefore, as inner shaft 130 rotates with respect to outer shaft 120 the overall outer dimension of coil 110 changes. For example, in FIG. 1A, coil 110 has an outer diameter D1 and loosely surrounds inner shaft 130. Rotating inner shaft 130 in a first direction relative to outer shaft 120 (via handle 141) as shown in FIG. 1B causes coil 110 to wrap more tightly around inner shaft 130 (collapse), thereby reducing coil 110 to a smaller outer diameter D2. Rotating inner shaft 130 in the opposite direction relative to outer shaft 120 as shown in FIG. 1C causes coil 110 to unwind further from inner shaft 130 (expand), resulting in an enlarged outer diameter D3.

Coil 110 can be formed from any material that enables this winding/unwinding. For example, in various embodiments, coil 110 can be formed from a resilient, flexible material, such as a shape memory material (e.g., Nitinol) or other spring material (e.g., spring steel), a biomaterial (e.g., polyaryletheretherketone (PEEK)), or various combinations of materials, such as a coil coated with a biostable material (e.g., Nitinol coated with PEEK), a coil coated with a bioresorbable material (e.g., Nitinol coated with one or more of a polylactide, polyglicolide, polycaprolactone, hydrogel, or protein polymer). In various other embodiments, drugs (e.g., antibiotics, antimicrobials, anti-inflammatories, corticosteroids, or pain-relieving agents) and/or bioactive agents (e.g., hydroxyappatite, anti-microbial silver, bone morphogenic protein (BMP), or bone-promoting biologics/biomaterials) can be used as a coating on the coil or can be embedded in the coil surface/coating

In various other embodiments, coil 110 can be formed from a material that plastically deforms in response to the rotational input provided by rotational actuator 140 (e.g., gold or aluminum oxide). In various other embodiments, coil 110 can be formed from a material that elastically deforms up to a certain point, and plastically deforms upon further expansion (e.g., Ti-6Al-4V and Co—Cr alloys). Various other material selections and/or combinations will be readily apparent.

Note also that minimally invasive procedures such as kyphoplasty are typically performed under fluoroscopy, so that the physician can at least have some visual indication of the surgical activity within the patient. Therefore, in one embodiment, coil 110 can be formed from, or can include, radiopaque material(s). Additionally, in various other embodiments, radiopaque markers can be placed at various locations on outer shaft 120 and/or inner shaft 130 to facilitate positioning of expandable bone tamp 100 in the patient.

In this manner, the size of coil 110 can be manually controlled to enable use of expandable bone tamp 100 in a surgical procedure, as described in greater detail below. This controlled expansion allows coil 110 to exert a significant amount of force on any surrounding bone and/or tissue to effect the surgical procedure. Note that while coil 110 is depicted as a being formed from a flat ribbon of material for exemplary purposes, the specific construction of coil 110 can take any form, depending on the desired performance of expandable bone tamp 100.

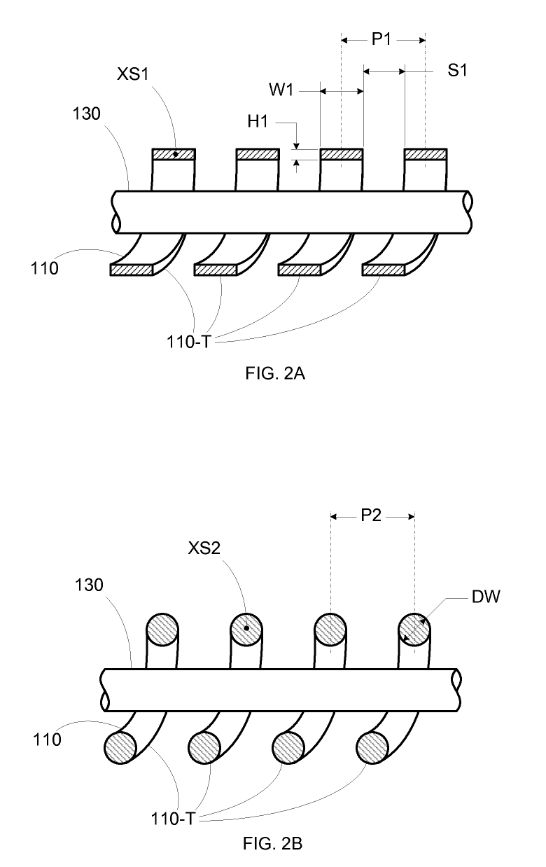

For example, FIG. 2A shows a partial cross-sectional view of coil 110 that depicts an exemplary rectangular cross section for the coil turns (loops) 110-T. Each turn 110-T has a material cross section XS1 (perpendicular to the direction of the loop) in the shape of a rectangle having width W1 and height H1. Note that a coil pitch P1 (i.e., the distance between centerlines of adjacent coil turns 110-T) and the spacing S1 between adjacent turns 110-T will vary based on how “wound” or “unwound” coil is by the rotational orientation of inner shaft 130 with respect to outer shaft 120 (not shown).

FIG. 2B shows another partial cross-sectional view of coil 110 that depicts an embodiment in which coil turns 110-T have round material cross sections XS2 with diameters DW, and a turn pitch P2. Various other material cross section shapes for turns 110-T will be readily apparent (e.g., triangular, trapezoidal, or oval, among others).

The particular cross-sectional shape can be selected based on the desired use of expandable bone tamp 100. For example, a rectangular cross section for coil turns 110-T such as shown in FIG. 2A can beneficially provide a relatively “flat” outer surface of coil 110 as it is deployed (expanded), which may enhance the ability of coil 110 to compress, rather than cut through, surrounding bone as it is expanded. However, a round cross-sectional shape for turns 110-T as shown in FIG. 2B may be easier to manufacture, while still providing acceptable performance.

Note that while the material cross sections XS1 and XS2 shown in FIGS. 2A and 2B, respectively, are depicted as being substantially constant, in various other embodiments, the material cross sections can vary (i.e., different dimensions and/or shapes) for different turns 110-T in coil 110 (and in some embodiments, vary even within individual turns 110-T). For example, increasing the height H1 for certain coil turns 110-T in coil 110 of FIG. 2A can cause those turns to expand less than turns with a thinner material cross section (i.e., smaller height H1). Likewise, the material cross-section diameter DW of turns 110-T in FIG. 2B can be varied to adjust the expansion properties of those coil turns. In addition, in various other embodiments, the turn pitch of coil 110 can be varied over the length of coil 110 to create a desired expansion profile.

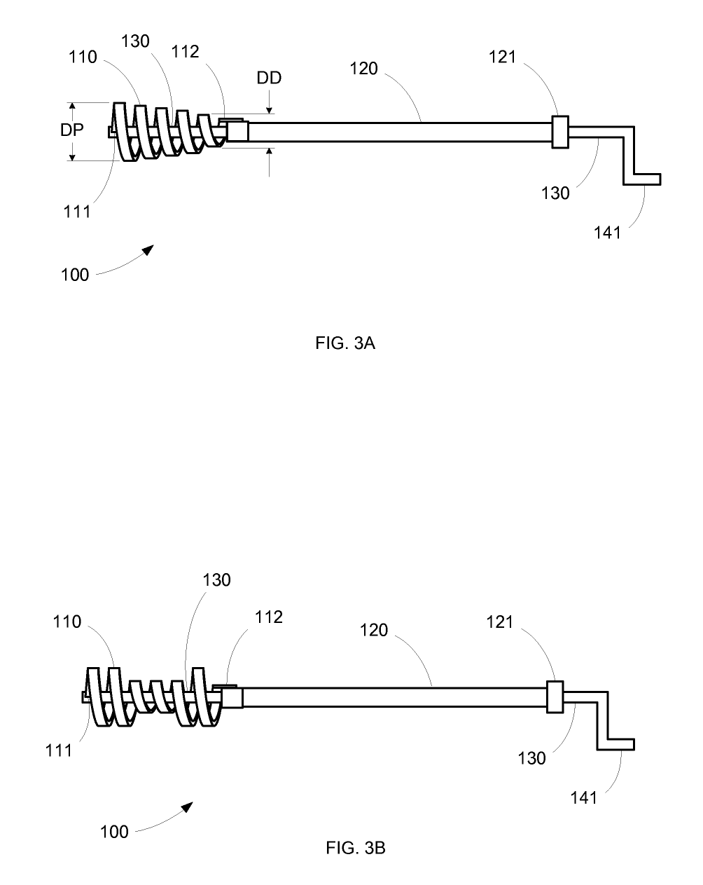

Note further that while coil 110 shown in FIGS. 1A-1C and 2A-2B are depicted as having a substantially constant outer diameter (e.g., diameters D1, D2, and D3), in various other embodiments, coil 110 can be configured to expand in a non-cylindrical manner. For example, FIG. 3A shows an embodiment of expandable bone tamp 100 in which coil 110 expands more distally than proximally, thereby creating something of a taper from distal coil end 111 to proximal coil end 112. As noted above with respect to FIG. 2A, in one embodiment, this type of expansion profile could be created by increasing the turn thickness (H1) or width (W1) of coil 110 from distal end 111 to proximal end 112. FIG. 3B shows another embodiment of expandable bone tamp 100 that exhibits a “dumbbell” shape (i.e., the proximal and distal ends of coil 110 expand more than its middle section). Various other expansion profiles for coil 110 will be readily apparent (e.g., ovoid, conical, asymmetrical).

Returning to FIG. 1C, it can be seen that the use of coil 110 in expandable bone tamp 100 provides a great deal of configuration flexibility. In some embodiments, coil 110 can be a permanent part of expandable bone tamp 100. For example, after bone manipulation, coil 110 could be wound back on to inner shaft 130 (e.g., as shown in FIG. 1B) and withdrawn with the rest of expandable bone tamp 100 from the patient. However, in various other embodiments, coil 110 can be detachable from expandable bone tamp 100 (e.g., to be left as an implant in the patient).

For example, FIG. 1D shows an embodiment of expandable bone tamp 100 in which end 111 of coil 110 is coupled to inner shaft 130 via a releasable connector 131, and end 112 of coil 110 is coupled to outer shaft 120 via a releasable connector 122. Note that while releasable connectors 131 and 122 are depicted as slots for exemplary purposes, in various other embodiments, any type of releasable coupling mechanism could be used (e.g., remotely actuated clips/clamps, or breakaway connections, among others).

Therefore, once coil 110 is expanded to a desired size, inner shaft 130 and outer shaft 120 can be pulled away (disconnected) from coil 110. In some embodiments, coil 110 could be made from a material that retains the last size and/or shape imposed by inner shaft 130 and outer shaft 120 (i.e., a plastically deforming material). In other embodiments, coil 110 could be made from a material that assumes a desired shape upon release from the rest of expandable bone tamp 100 (e.g., a shape memory material that causes coil 110 to contract or continue to try to expand). Various other material configurations will be apparent.

FIG. 1E shows another embodiment of a coil 110 that can be detached from expandable bone tamp 100. In FIG. 1E, coil 110 remains connected to a portion 130A of inner shaft 130 and a portion 120A of outer shaft 120. A releasable connection formed by connection features 123 and 124 on outer shaft portion 120A and outer shaft 120, respectively, allows outer shaft portion 120A to be detached from the rest of outer shaft 120. Likewise, a releasable connection formed by connection features 133 and 134 on inner shaft portion 130A and inner shaft 130, respectively, allows inner shaft portion 130A to be detached from the rest of inner shaft 130. Note that while the releasable connection for outer shaft 120 is depicted as a threaded connection for exemplary purposes, and the releasable connection for inner shaft 130 is depicted as a slot/blade connection for exemplary purposes, any type of releasable connection can be used for either outer shaft 120 and inner shaft 130.

In one embodiment, outer shaft portion 120A can include a holding mechanism 121A for maintaining the rotational orientation of inner shaft portion 130A with respect to outer shaft portion 120A. Holding mechanism 121A can be substantially similar to, or take the place of, holding mechanism 121 described above with respect to FIGS. 1A-1C to enable coil 110 to maintain its deployed shape after it has been detached from the rest of expandable bone tamp 100.

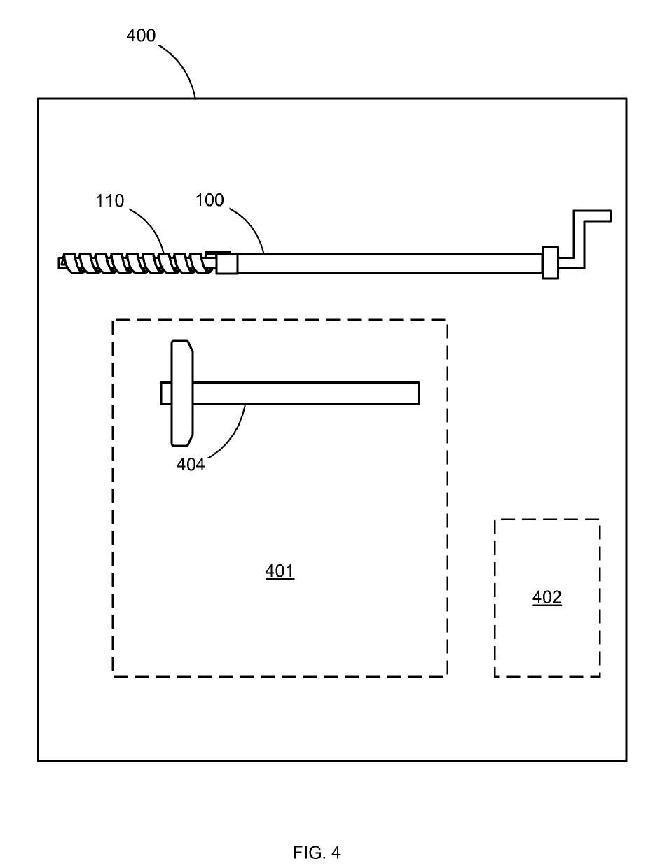

FIG. 4 shows a diagram of a kit 400 for use in performing a surgical procedure, such as a kyphoplasty procedure, as described in greater detail below. Kit 400 includes an expandable bone tamp 100 that includes an expandable coil 110 (e.g., as described above with respect to FIGS. 1A-1E, 2A-2B, and 3A-3B). In various embodiments, kit 400 can further include optional additional instruments 401, such as a cannula 404 sized to receive expandable bone tamp 100, an introducer, guide pin, drill, curette, and/or access needle, among others (only cannula 404 is shown for clarity). In various other embodiments, kit 400 can further include optional directions for use 402 that provide instructions for using expandable bone tamp 100 and optional additional instruments 401 (e.g., instructions for performing a kyphoplasty procedure using expandable bone tamp 100 and optional additional instruments 401).

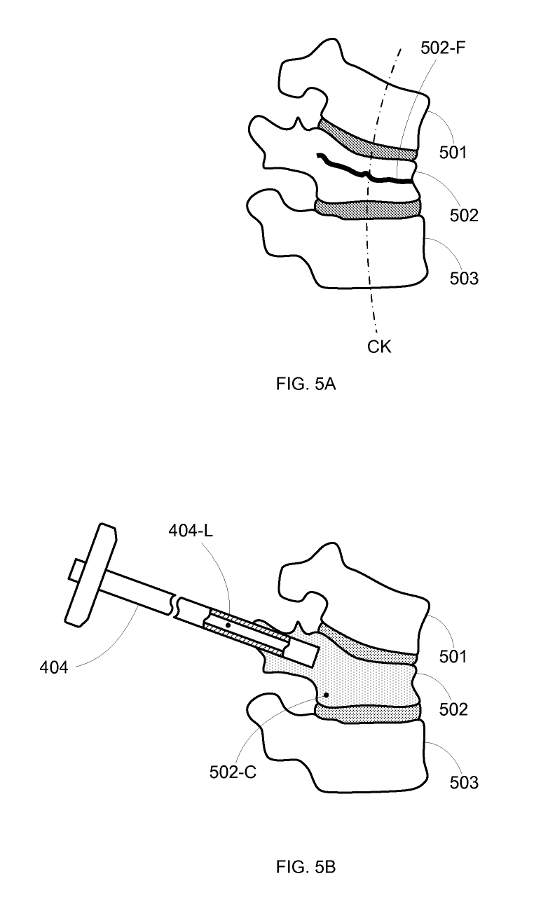

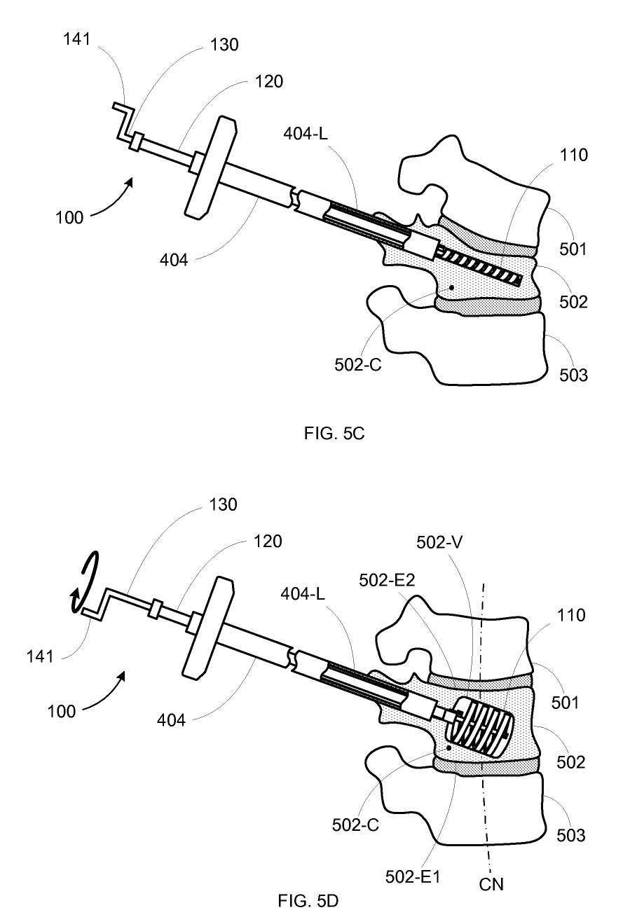

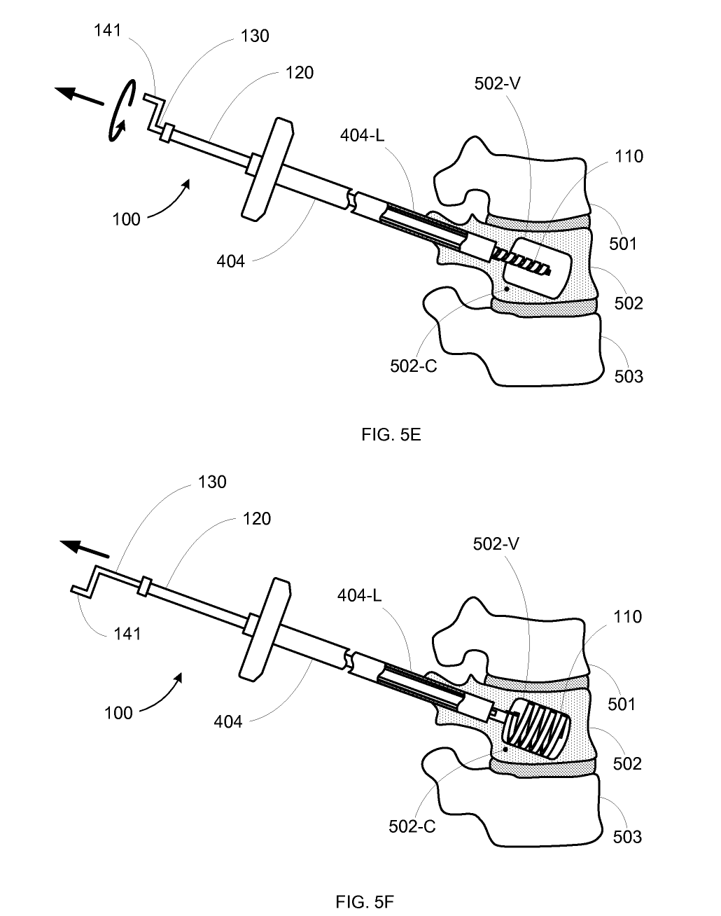

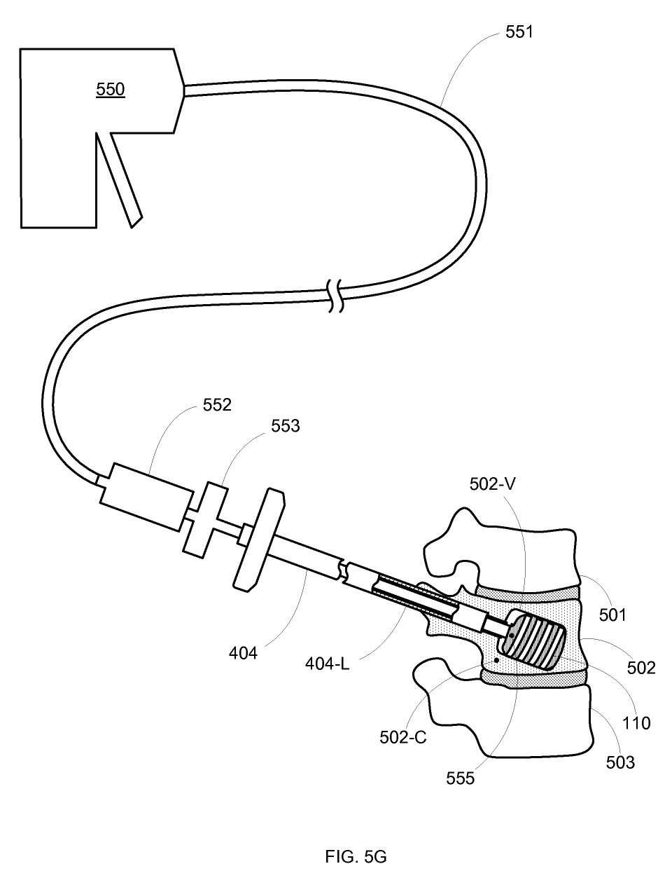

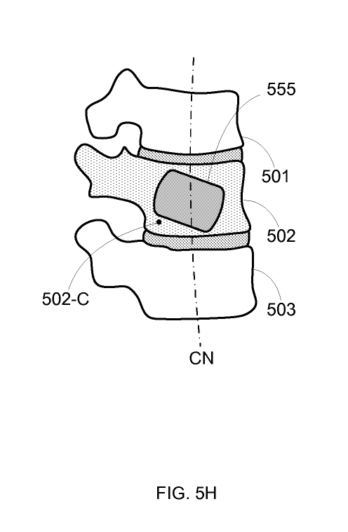

FIGS. 5A-5H show an exemplary kyphoplasty procedure using an expandable bone tamp 100 that incorporates a coil 110. FIG. 2A shows a portion of a human vertebral column having vertebrae 501, 502, and 503. Vertebra 502 has collapsed due to a vertebral compression fracture (VCF) 502-F that could be the result of osteoporosis, cancer-related weakening of the bone, and/or physical trauma. The abnormal curvature CK of the spine caused by VCF 502-F can lead to severe pain and further fracturing of adjacent vertebral bodies.

FIG. 5B shows a cannula 404 being positioned next to the target surgical location, which in this case is the cancellous bone structure 502-C within fractured vertebra 502. In this manner, a percutaneous path to vertebra 502 is provided via an interior lumen 404-L of cannula 404. Typically, cannula 404 is docked into the exterior wall of the vertebral body (using either a transpedicular or extrapedicular approach) using a guide needle and/or dissector, after which a drill or other access tool (not shown) is used to create a path further into the cancellous bone 502-C of vertebra 502. However, any other method of cannula placement can be used to position cannula 404.

Then in FIG. 5C, an expandable bone tamp 100 (as described above with respect to FIGS. 1A-1E, 2A-2B, and 3A-3B) is placed into cannula 404. During this initial placement of expandable bone tamp 100 into vertebra 502, coil 110 is would closely around inner shaft 130 (e.g., as described with respect to FIG. 1B) to enable easy passage through interior lumen 404-L of cannula 404. Note that in one embodiment, a tightly wound coil 110 can be used as a drill to actually create its own placement channel within cancellous bone 502-C.

Next, as shown in FIG. 5D crank handle 141 is used to rotate inner shaft 130 relative to outer shaft 120, thereby enlarging coil 110. As coil 110 expands, it compacts and/or displaces cancellous bone 502-C while creating a cavity 502-V. In addition, the powerful expansion force generated by coil 110 pushes apart endplates 502-E1 and 502-E2 of vertebra 502, thereby partially or fully restoring the original height of vertebra 502. As a result, a normal spinal curvature CN can be achieved to prevent the physical problems that would otherwise be associated with the kyphosis caused by the fracture of vertebra 502.

Note that while the expansion of coil 110 is described above as a single operation, in certain circumstances, it may be desirable to perform the expansion in incremental steps or as a series of expansions and contractions. For example, to break apart a fractured vertebra that has healed in the fractured state, it may be beneficial to expand coil 110 to a particular size or until a particular resistance is noted, pause for some amount of time, expand coil 110 a bit more, and so forth. In this manner, the expansion forces from coil 110 can be applied gradually to the vertebral body, thereby minimizing the risk of sudden uncontrolled re-fracture.

In another example, a fractured vertebral body may have regions of hard cancellous bone that initially resist compression. In such a case, coil 110 can be repeatedly expanded and contracted (collapsed) to eventually break down the harder portions of cancellous bone. Various other expansion techniques will be readily apparent for different bone conditions.

Once a desired cavity 502-V and/or height restoration of vertebra 502 is achieved, expandable bone tamp 100 can either be removed completely from vertebra 502, as shown in FIG. 5E, or coil 110 can be left behind in cavity 502-V, as shown in FIG. 5F. In FIG. 5E, coil 110 is re-wrapped around inner shaft 130 by turning crank handle 141 in the direction opposite to the deployment direction, after which expandable bone tamp 100 can be removed through cannula 404. In FIG. 5F, coil 110 is detached from the rest of expandable tamp 100 (e.g., as described with respect to FIG. 1D or 1E) and remains within cavity 502-V.

In either case, once expandable bone tamp 100 is removed from cannula 404, cavity 502-V can be filled with bone filler material 555 (e.g., PMMA), as shown in FIG. 5G. A delivery nozzle 553 is inserted through cannula 404 and into cavity 502-V, and is used to direct bone filler material 555 into cavity 502-V. Note that coil 110 is shown remaining in cavity 502-V during this fill operation for exemplary purposes. In various other embodiments, coil 110 can be removed from cavity 502-V prior to delivery of bone filler material 555, as described with respect to FIG. 5E.

As shown in FIG. 5G, in one embodiment, a quantity of bone filler material 555 can be housed in a cartridge 552 attached to delivery nozzle 553. A hydraulic actuator 550 can then be used to remotely express bone filler material 555 from cartridge 552 via a hydraulic line 551 (e.g., cartridge 552 can include a piston that is driven by the hydraulic pressure supplied by hydraulic line 551).

Note, however, that in various other embodiments, bone filler material 555 can be delivered to cavity 502-V in any number of different ways (e.g., a high pressure cement delivery pump that delivers the cement to nozzle 553 through a flexible line, or a syringe or other delivery device filled with bone filler material 555 that is attached directly to nozzle 553), In addition, in various other embodiments, bone filler material 555 can be delivered in multiple portions of the same or different materials (e.g., a bone cement followed by a biologic agent).

Once the filling operation is complete, delivery nozzle 553 and cannula 404 are removed from vertebra 502 (and the patient's body) as shown in FIG. 5H. Upon hardening, bone filler material 555 provides structural support for vertebra 502, thereby substantially restoring the structural integrity of the bone and the proper musculoskeletal alignment of the spine. As shown in FIG. 5H, due to the restoration of height in fractured vertebra 502, the abnormal curvature CK shown in FIG. 5A is corrected to a normal curvature CN. In this manner, the pain and attendant side effects of a vertebral compression fracture can be addressed by a minimally invasive kyphoplasty procedure.

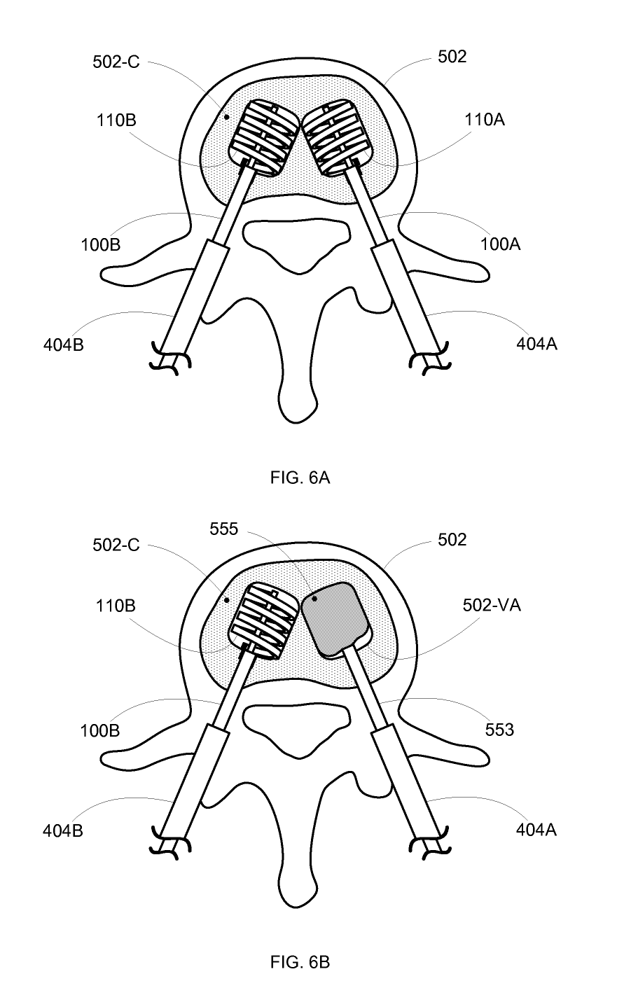

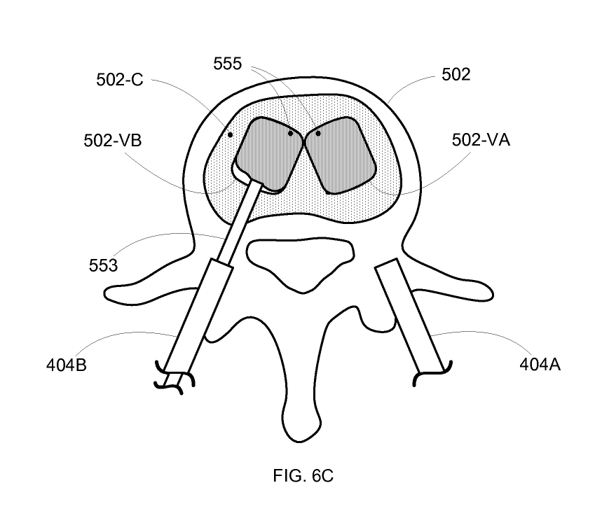

Note that although the kyphoplasty procedure described with respect to FIGS. 5A-5H makes use of a single expandable bone tamp 100 for clarity, in various other embodiments, any number of expandable bone tamps can be used. For example, FIG. 6A shows a top view of vertebra 502 in which a bilateral transpedicular procedure is being performed using two expandable bone tamps 100A and 1008. Both coils 110A and 110B of expandable bone tamps 100A and 1006, respectively, have been expanded (e.g., as described with respect to FIG. 5D). This bilateral approach can help to ensure that any height restoration of vertebra 502 occurs evenly.

Next, in FIG. 6B, expandable bone tamp 100A is removed from vertebra 502, and the cavity 502-VA that remains in vertebra 502 is filled with bone filler material 555 via a delivery nozzle 553 (e.g., as described with respect to FIG. 5G). During this filling operation, coil 110B remains deployed within vertebra 502 to ensure that any height restoration is maintained during this fill operation.

Once bone filler material 555 in cavity 502-VA is sufficiently hardened, expandable bone tamp 100B can be removed from cannula 404B, and the remaining cavity 502-VB can be filled, this time with the hardened bone filler material 555 in cavity 502-VA providing the endplate support, as shown in FIG. 6C. Note that in various other embodiments, a similar sequential fill operation can be performed with coils 110A and/or 110B left within cavities 502-VA and 502-VB, respectively, after expandable bone tamps 100A and 100B, respectively, are withdrawn from vertebra 502.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

1. A device for performing a surgical procedure, the device comprising:
an outer shaft;
an inner shaft rotatably disposed within the outer shaft;
a coil having a first end and a second end, the first end being coupled to the outer shaft, and the second end being coupled to the inner shaft; and
a rotational actuator for rotating the inner shaft relative to the outer shaft.

2. The device of claim 1, wherein rotating the inner shaft in a first direction relative to the outer shaft causes the coil to expand, and
wherein rotating the inner shaft in a second direction relative to the outer shaft causes the coil to collapse.

3. The device of claim 1, wherein the rotational actuator comprises at least one of a crank handle coupled to the inner shaft, a motor, a lead screw, a ball screw, a cable, and a gear train.

4. The device of claim 1, further comprising a holding mechanism for controllably maintaining a rotational orientation between the inner shaft and the outer shaft.

5. The device of claim 4, wherein the holding mechanism comprises at least one of a clamp, a ratchet, and a shaft collar.

6. The device of claim 1, wherein the coil comprises at least one of a shape memory material, a spring material, a Co—Cr alloy, a biostable material, a bioresorbable material, a drug, and a bioactive agent.

7. The device of claim 1, wherein the coil comprises a plurality of turns; each of the plurality of turns having a material cross section in the shape of one of one of a rectangle, a circle, an oval, a triangle, and a trapezoid.

8. The device of claim 1, wherein the coil comprises:
a first turn having a first material cross section; and
a second turn having a second material cross section,
wherein the first material cross section is different than the second material cross section in at least one of size and shape.

9. The device of claim 1, wherein the coil comprises:
a first turn; and
a second turn,
wherein the first turn and the second turn expand to a first size and a second size when the inner shaft is rotated in a first direction relative to the outer shaft, the second size being larger than the first size.

10. The device of claim 9, wherein the coil comprises a third turn,
wherein the third turn expands to a third size when the inner shaft is rotated in the first direction relative to the outer shaft, the third size being larger than the second size.

11. The device of claim 1, wherein the first end of the coil is coupled to the outer shaft by a first releasable connector, and
wherein the second end of the coil is coupled to the inner shaft by a second releasable connector.

12. The device of claim 1, wherein the first end of the coil is coupled to a first portion of the outer shaft, the first portion of the outer shaft being detachable from the outer shaft, and
wherein the second end of the coil is coupled to a first portion of the inner shaft, the first portion of the inner shaft being detachable from the inner shaft.

13. A system for performing a surgical procedure, the system comprising:
a cannula defining an interior lumen; and
an expandable bone tamp comprising:
an outer shaft;
an inner shaft rotatably disposed within the outer shaft; and
a coil having a first end and a second end, the first end being coupled to the outer shaft, and the second end being coupled to the inner shaft,
wherein the coil is sized to pass through the interior lumen when wound around the inner shaft by rotating the inner shaft in a first direction relative to the outer shaft, and
wherein the coil can be enlarged beyond a diameter of the interior lumen by rotating the inner shaft in a second direction relative to the outer shaft.

14. The system of claim 13, further comprising a rotational actuator for rotating the inner shaft with respect to the outer shaft, the rotational actuator comprising at least one of a crank handle coupled to the inner shaft, a motor, a lead screw, a ball screw, a cable, and a gear train.

15. The system of claim 13, wherein the coil comprises at least one of a shape memory material, a spring material, polyaryletheretherketone, a Co—Cr alloy, a biostable material, a bioresorbable material, a drug, and a bioactive agent.

16. The system of claim 13, wherein the coil comprises a plurality of turns, each of the plurality of turns having a material cross section in the shape of one of one of a rectangle, a circle, an oval, a triangle, and a trapezoid.

17. The system of claim 13, wherein the coil comprises:
a first turn; and
a second turn,
wherein the first turn and the second turn expand to a first size and a second size when the inner shaft is rotated in a first direction relative to the outer shaft, the second size being larger than the first size.

18. The system of claim 13, wherein the coil is detachable from the expandable bone tamp.

19. A method for performing a surgical procedure, the method comprising:
placing a coil in a bone, wherein a first end of the coil is connected to an outer shaft, and wherein a second end of the coil is connected to an inner shaft, the inner shaft being rotatably disposed in the outer shaft;
creating a cavity within the bone by rotating the inner shaft in a first direction relative to the outer shaft to increase an outer diameter of the coil; and
filling the cavity with a bone filler material.

20. The method of claim 18, wherein placing the coil in a bone comprises docking a cannula with the bone, the cannula defining an interior lumen, and positioning the coil in the bone through the interior lumen, and
wherein creating the cavity within the bone further comprises rotating the inner shaft in a second direction relative to the outer shaft to decrease the outer diameter of the coil and then withdrawing the coil through the interior lumen.

21. The method of claim 18, wherein filling the cavity with the bone filler material comprises delivering the bone filler material to the cavity while the coil is within the cavity.
